(19) United States
(12) Patent Application Publication
(10) Pub. No.: US 2022/0011323 A1
(43) Pub. Date: Jan. 13, 2022

(54) METHOD FOR DIAGNOSIS AND TREATING PERIPHERAL NEUROPATHIES

(71) Applicants: ASSISTANCE PUBLIQUE -HÔPITAUX DE PARIS, Paris (FR); SORBONNE UNIVERSITÉ, Paris (FR)

(72) Inventors: Olivier BOURRON, Vitry sur Seine (FR); Joe-Elie SALEM, Paris (FR); Agnès HARTEMANN, Clamart (FR)

(21) Appl. No.: 17/294,007

(22) PCT Filed: Nov. 14, 2019
(86) PCT No.: PCT/EP2019/081251

§ 371 Date: May 14, 2021

(30) Foreign Application Priority Data
Nov. 15, 2018 (EP) 18306503.6

(51) Int. Cl. G01N 33/68

(52) U.S. Cl. CPC G01N 33/6896; G01N 2800/28; G01N 2800/042

(57) ABSTRACT

The present invention relates to MGP as a new serum marker useful by itself or in combination with other markers for diagnosis of peripheral neuropathies, in particular in diabetic patients. The invention is also drawn to diagnosis kits for the implementation of this method.

The present invention relates to MGP as a new serum marker useful by itself or in combination with other markers for diagnosis of peripheral neuropathies, in particular in diabetic patients. The invention is also drawn to diagnosis kits for the implementation of this method.

Peripheral neuropathies, a result of damage to the peripheral nerves, often cause weakness, numbness and pain, usually in hands and feet. It can also affect other areas of the body.

Diabetic peripheral neuropathy (DPN) is a frequent (the most common) complication of diabetes. It affects about 10 to 15% of patients with type 2 diabetes at diagnosis and up to 50% after 10 years of disease duration (Pop-Busui et al. Diabetes Care. 2017; 40(1):136-54). Diabetic neuropathy is associated to high morbidity and mortality, because of increased risk for foot ulceration and amputation, and for poor quality of life and depression. So, it is related to high healthcare costs. Distal symmetric sensorimotor polyneuropathy is the most common form of diabetic neuropathy. Its diagnosis is clinical, and is based on interviewing but also on physical examination, due to up to 50% of asymptomatic forms. In 2017, ADA guidelines recommended an annual screening by assessment of either temperature or pinprick sensation and vibration sensation (Pop-Busui et al, op. cit.). Mechanisms involved in diabetic neuropathy are not clearly understood. The main hypothesis is that chronic glucotoxicity and lipotoxicity lead to oxidative stress, inflammation, and mitochondrial dysfunction and finally to nerve damage with neuron degeneration and demyelination.

Early detection of DPN in at-risk individuals and in those with prediabetes (PD) or type 2 diabetes (T2D) allows for potential better management through optimal intervention and lifestyle changes. In particular, early detection of DPN makes it possible to reduce the number of feet amputations.

It can be performed by analysis of walking patterns, using plantar pressure sensors and smartphones built-in accelerometers, using a 128 Hz tuning fork to assess vibration perception, 1 g and 10 g monofilaments with a standard lab testing table to evaluate sensation perception, corneal confocal microscopy.

However, these methods may require training of the physicians to be performed in a reliable way, or expensive material.

WO2009023140 relates to detection and/or monitoring of inflammatory neuropathy using markers that specifically indicate the presence of inflammatory neuropathy, for example, allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof.

WO2016131993 relates to a new indication of vitamin K for improving microvascular integrity and capillary structure and function, thus preventing, mitigating, counteracting or curing diseases associated with impaired capillary morphology including the glycocalyx or capillary dysfunction.

Gomez et al (BMJ Case Rep. 2016 Jun. 17; 2016) report the case of a 60-year-old man who had monoclonal gammopathy of undetermined significance (MGUS)-associated neuropathy and for whom warfarin showed a remarkable impact on symptoms and quality of life.

There is thus a need to develop new diagnosis tests, reliable, easy to perform, that are able to detect peripheral neuropathy at an early stage.

The inventors demonstrated that dosing the amount of a specific form of Matrix gla protein (MGP) in a biological sample (blood or plasma) of a patient makes it possible to detect presence of peripheral neuropathy.

This diagnosis is even more accurate when other markers are used.

It is possible to first perform dosage of MGP and then perform the more accurate method only for patients with an elevated level of MGP.

Furthermore, the inventors propose to inhibit the activity of MGP to treat peripheral neuropathies, in particular diabetic peripheral neuropathies.

Matrix gla protein (MGP) is a 84 amino acids protein containing five gamma-carboxyglutamic (gla) residues. This mature protein is obtained from a 103 protein after excision of the first 19 N-terminal amino acids (signal peptide).

Its accession number is NP_000891 (NCBI Reference Sequence).

It was initially isolated from bone tissue, but it is also expressed by chondrocytes, vascular smooth muscle cells and endothelial cells. It is implicated in inhibition of arterial calcification. This inhibition requires the active form of MGP (Schurgers et al., J Thromb Haemost J T H. 2007; 5(12):2503-11), which is obtained after a gammaglutamyl carboxylation of glutamic acid (glu) residues, dependent of vitamin K (Engelke et al., Biochim Biophys Acta. 1991; 1078(1):31-4), and a phosphorylation of serine residues (Price et al., Protein Sci Publ Protein Soc. 1994; 3(5):822-30).

In 2005, a mutation of MGP was described, associated with neurological manifestations, abnormalities of brain's white matter and optic nerve atrophy, in addition to typical manifestations of Keutel syndrome (Hur et al, Am J Med Genet A. 2005; 135(1):36-40). Goritz and al have demonstrated that MGP is expressed by neurons, and is regulated by glial cells (Göritz et al., Glia. 15.08. 2007; 55(11):1108-22). Some studies revealed that MGP has differential expression of MGP in various diseases such as glioblastoma (Mertsch et al., BMC Cancer. 2009; 9:302), or Alzheimer disease (Santa-Maria et al., Neurosci Lett. 2010; 468(3):225-8).

The quality of a test is generally determined by drawing a Receiving Operating Characteristic (ROC) curve and measuring the Area Under Receiving Operating Characteristic curve (AUROC).

The ROC curve is drawn by plotting the sensitivity versus (1-specificity), after classification of the patients, according to the result obtained for the test, for different thresholds (from 0 to 1).

It is usually acknowledged that a ROC curve, the area under which has a value superior to 0.7, is a good predictive curve. The ROC curve has to be acknowledged as a curve allowing prediction of the quality of a test. It is best for the AUROC to be as closed as 1 as possible, this value describing a test which is 100% specific and sensitive.

It is reminded that

(1) sensitivity is the probability that the diagnosis is positive in individuals having the phenotype sought (detection of true positives): the test is positive if the patient is having the phenotype. The sensitivity is low when the number of false negatives is high. The sensitivity is calculated by the formula SE=(number of individuals having the phenotype in whom the sign is present)/(number of individuals having the phenotype in whom the sign is present+number of individuals having the phenotype in whom the sign is absent).

(2) specificity is the probability that the diagnosis is negative in the individuals not having the phenotype sought (non-detection of true negatives): the test is negative if the patient is not suffering from the disease. The specificity is low when the number of false positives is high. The specificity is calculated by the formula SP=(number of individuals not having the phenotype in whom the sign is absent)/(number of individuals not having the phenotype in whom the sign is absent+number of individuals not having the phenotype in whom the sign is present).

(3) Positive predictive value (PPV): is the probability of having the disease if the diagnostic test is positive (i.e. that the patient is not a false positive): the patient is having the phenotype if the test is positive. The positive predictive value is calculated by the formula PPV=(number of individuals having the phenotype in whom the sign is present)/(number of individuals having the phenotype in whom the sign is present+number of individuals not having the phenotype in whom the sign is present).

(4) Negative predictive value (NPV): is the probability of not having the disease if the diagnostic test is negative (that the patient is not a false negative): the patient is not having the phenotype if the test is negative. The negative predictive value is calculated by the formula NPV=(number of individuals not having the phenotype in whom the sign is absent)/(number of individuals not having the phenotype in whom the sign is absent+number of individuals having the phenotype in whom the sign is absent)

Generally, a diagnosis (or prognosis) method comprises

i. a step of gathering information from the patient

ii. a step of comparing said information with regards to thresholds

iii. a step of deducing, from the difference between the patient's information and the threshold, whether the patient has a specific disease, the stage of the patient's disease, or whether the patient's state will evolve to a given state.

Some methods, such as the ones disclosed in the present application, shall also include a step i.a), which comprise the steps of modifying the information obtained from the patient in order to obtain a new type of information, which is the one that is then compared to the standards in step ii. Such modification is the combination of the values of variables in a function, and obtaining an end value.

It is further to be noted that the mere measurement of the values of levels of markers in the plasma or serum of a patient and the combination thereof in an algorithm as herein disclosed is part of a method but only provides an intermediate result (an end value or index) that would then to be compared to a reference index (threshold), in order to actually be able to pose the diagnostic.

It is also to be noted that the tests herein disclosed are not “gold-standard” tests, in the sense that the output (index calculated by the formulas herein disclosed) isn't a definitive answer as to the state of the patient. Indeed, these tests are based on statistics and there may thus be false-positive or false-negative results, which is the reason why the specific experience of the physician in interpreting the index is of importance for making the prognosis and deciding which kind of follow up is to be made to ne made for each patient.

However, depending on the specificity, sensitivity, positive predictive value and negative predictive value of the tests, for various thresholds, step iii as disclosed above is not direct and immediate from step ii, as the physician must interpret the result from the clinical and general context to be able to reach a conclusion. These tests are of great interest in provided a help to the physician when investigating a clinical case.

The invention thus relates to a method for determining the presence of peripheral neuropathy in a patient, comprising the steps of:

a) Determining the level of dephosphorylated uncarboxylated MGP in a sample from said patient and optionally of other clinical markers

b) when other markers are used, combining the values obtained in a) through a function in order to obtain an end result

c) Comparing the level of dephosphorylated uncarboxylated MGP measured in step a) or of the end value obtained in step b) to a predetermined threshold

wherein the patient has peripheral neuropathy if the level of dephosphorylated uncarboxylated MGP measured in step a) or of the end value obtained in step b) is higher than the threshold.

In a preferred embodiment, the peripheral neuropathy is a diabetic peripheral neuropathy. It is especially true for a patient with type 2 diabetes.

This function is particularly adapted to diagnose the presence of peripheral neuropathy with a NDS≥6 (see below).

It is to be noted that the method can be used to determine that a patient has not a peripheral neuropathy, when the end value (or the level of dp-ucMGP is lower than the threshold. This is particularly interesting to exclude these patients from other tests. This is in particular true for the functions disclosed in the examples, which have high specificity and Negative Predictive Value.

If the value is higher than the threshold, treatment can be provided to the patient, or further extensive investigation may be performed.

In a first aspect, the invention relates to a method for determining the presence of diabetic peripheral neuropathy in a patient, comprising the steps of:

a) Measuring the level of dephosphorylated uncarboxylated MGP in a sample from said patient

b) Comparing the level measured in step a) to a threshold

wherein the patient has diabetic peripheral neuropathy if the level of dephosphorylated uncarboxylated MGP(dp-ucMGP) is higher than the threshold.

This method is performed ex vivo or in vivo, and makes it possible to pose the diagnosis of diabetic peripheral neuropathy.

This diagnosis may be strengthened by other evidence, such as loss of sensibility in some organs, or by response to other tests already known in the art.

In a preferred embodiment, the level (amount) of dephosphorylated uncarboxylated MGP in step a) is measured by ELISA assay. Using a dual-antibody ELISA assay is particularly adapted in this case, in order to specifically detect both the dephosphorylated and uncaborxylated form of the protein. One shall thus preferably use an antibody that is able to discriminate the dephosphorylated and the phosphorylated forms of the MGP. Another antibody, able to discriminate the carboxylated and uncarboxylated forms of the MGP is also used in this dual-antibody ELISA assay.

Such antibodies are known in the art, and are disclosed in particular in Cranenburg et al (Thromb Haemost. 2010 October; 104(4):811-22), and in the list of references of this publication.

In particular the capture antibody may be directed against the dephosphorylated MGP sequence 3-15 and the detecting antibody directed against the uncarboxylated MGP sequence 35-49.

In another embodiment, though, the detecting antibody may be directed against the dephosphorylated MGP sequence 3-15 and the capture antibody directed against the uncarboxylated MGP sequence 35-49.

Such antibodies are in particular available from VitaK BV, Maastricht, the Netherlands.

When the amount of dp-ucMGP is a higher than a specific threshold, the diagnosis of presence of peripheral neuropathy may be made. In a preferred embodiment, the threshold is 700 pmol/l.

In another embodiment, other markers are combined with the level of dp-ucMGP.

In this embodiment, the invention is drawn a method for diagnosis of liver fibrosis and/or presence of liver necroinflammatory lesions in a patient comprising the steps of:

a) combining the values of clinical markers through a function in order to obtain an end value and,

b) comparing the end value to a predetermined value (threshold)

b) determining the presence of peripheral neuropathy in said patient, if the end value of said function is higher than the predetermined value.

wherein one of said biochemical markers is dp-uc MGP.

This method is thus performed in vitro or ex vivo. In a specific embodiment, the method also comprises the step of measuring or recovering the values of the clinical markers before step a).

In another embodiment, the invention relates to a method for treating a patient with peripheral neuropathy, comprising the steps of performing a diagnosis method as above and providing appropriate treatment and care to the patient if the end result is higher than the predetermined threshold.

In this embodiment, the other markers are preferably selected from the group consisting of height of the patient (m), total cholesterol (mmol/l), presence of insulin treatment (yes=0; no=1), presence of retinopathy treated with laser (yes=0; no=1), presence of a albumin/creatinine (mg/mmol) ratio>3 (yes=0; no=1), glycated hemoglobin (HbA1c) and presence of coronary arterial disease (yes=0; no=1).

In a specific embodiment, the other markers are height of the patient (m), total cholesterol (mmol/l), presence of insulin treatment (yes=0; no=1), presence of retinopathy treated with laser (yes=0; no=1), presence of a albumin/creatinine (mg/mmol) ratio>3.

In another embodiment, the other markers are preferably selected from the group consisting of height of the patient (m), total cholesterol (mmol/l), presence of insulin treatment (yes=0; no=1), presence of retinopathy treated with laser (yes=0; no=1).

In another embodiment, the other markers are preferably selected from the group consisting of height of the patient (m), presence of insulin treatment (yes=0; no=1), presence of retinopathy treated with laser (yes=0; no=1), presence of a albumin/creatinine (mg/mmol) ratio>3, glycated hemoglobin (HbA1c).

In the present invention, the function can be obtained in particular by

a) evaluating the presence of peripheral neuropathy in the patients of a cohort of patients, wherein the values of the appropriate markers variables are known for the patients

b) identifying by unidimensional analysis or multivariate analysis, among the markers, the value of which is known, the ones for which the values differ significantly between the groups of
i. patients with peripheral neuropathy
ii. patients without peripheral neuropathy

c) performing a logistic regression analysis, or any other statistical analysis, to assess and ponder the independent discriminative value of the markers identified in step b) for the presence of peripheral neuropathy

d) thereby obtaining the function herein used by combination of these identified independent factors.

In order to obtain a function that is as accurate as possible, the number of patients in the cohort should be as large as possible, indicating that it preferably comprises more than 50 patients, preferably more than 100 patients, preferably more than 200 patients, more preferably more than 500 patients, or even more than 1000 patients. As indicated, there is no upper limit for the number of patients, and the larger, the better.

In a specific embodiment, the function is

f1=1/(1+exp(−(−a*Height (m)+b*Total Cholesterol−c*dp-ucMGP(pM)+d*[Insulin treatment]+e*[retinopathy treated with laser]+f*[Albumin/creatinine ratio>3]+g))

with

8.3≤a≤8.9, more preferably 8.4≤a≤8.8, most preferably 8.5≤a≤8.7

0.45≤b≤0.75, more preferably 0.5≤b≤0.7, most preferably 0.55≤b≤0.65

1.02×10⁻³≤c≤1.06×10⁻³, more preferably 1.035×10⁻³≤c≤1.055×10⁻³, most preferably 1.04×10⁻³≤c≤1.05×10⁻³,

1.3≤d≤1.8, more preferably 1.4≤d≤1.7, most preferably 1.5≤4≤1.6

1.05≤e≤1.55, more preferably 1.15≤e≤1.45, most preferably 1.25≤e≤1.35

0.6≤f≤1.1, more preferably 0.7≤f≤1.0, most preferably 0.8≤f≤0.9

12.4≤g≤13.2, more preferably 12.6≤f≤13.0, most preferably 12.7≤f≤12.9

In this function,

Insulin treatment: in the presence of insulin treatment value=0; if not value=1

retinopathy treated with laser: in the presence of retinopathy treated with laser, value=0; if not, value=1

Albumin/creatinine ratio>3: if Albumin/creatinine ratio higher than 3, the value=0; if Albumin/creatinine ratio≤3, the value is 1.

In a specific embodiment,

a=8.600

b=0.616

c=1.045×10⁻³

d=1.541

e=1.312

f=0.863

g=12.805

Although the above-method can be used to detect any peripheral neuropathy, it is preferred when it is used to detect diabetic peripheral neuropathy, and in particular when the patient is a type 2 diabetic patient.

In particular, an elevated level of dp-ucMGP in the patient's sample may be indicative of presence of another peripheral neuropathy, such as chronic inflammatory demyelinating polyradiculoneuropathy, neuropathy associated with immunoglobulin M (IgM) monoclonal gammopathy and antibodies against myelin-associated glycoprotein (MAG), Charcot Marie Tooth Ia (CMT Ia) neuropathy, uremic neuropathy, or neuropathy caused by vitamin deficiency.

The diagnosis test and method herein disclosed is particularly effective in detecting peripheral neuropathies with a NDS (Neuropathy Disability Score) higher than 6. The NDS has been disclosed in Boulton (Management of diabetic peripheral neuropathy. Clin Diabetes 2005; 23).

The invention also relates to a kit for diagnosis of peripheral neuropathy (in particular diabetic peripheral neuropathy) in a patient, comprising

a) A container with first antibodies immobilized on at least one of its surfaces, wherein the first antibodies recognize a uncarboxylated MGP sequence

b) A solution comprising second antibodies recognizing the dephosphorylated MGP sequence, preferably bound to a label allowing detection of such antibodies.

Is also subject of the invention, a kit for diagnosis of peripheral neuropathy (in particular diabetic peripheral neuropathy) in a patient, comprising

a) A container with first antibodies immobilized on at least one of its surfaces, wherein the first antibodies recognize a dephosphorylated MGP sequence

b) A solution comprising second antibodies recognizing the uncarboxylated MGP sequence, preferably linked to a label allowing detection of such antibodies.

It is to be noted that recognition, by an antibody, of the dephosphorylated MGP sequence intends to indicate that such antibody recognizes an epitope within the MGP protein, that is associated with phosphorylation (in particular amino acids 3-15) and that said antibody will bind to this epitope if not phosphorylated, wherein the antibody doesn't bind to the epitope is phosphorylation has occurred.

Likewise, “antibodies recognizing the uncarboxylated MGP sequence” which recognize an epitope within the MGP protein, that is associated with carboxylation (in particular amino acids 35-49) and that said antibodies will bind to this epitope if not carboxylated, wherein the antibody doesn't bind to the epitope if carboxylation has occurred.

The detecting (second) antibodies are intended to allow detection of such bound antibodies and thereby presence and quantification of the dp-ucMGP. Such antibodies are modified by classical methods that are known in the art for revelation in ELISA assays. Such antibodies may thus be linked to biotin (biotinylated antibodies), dyes, enzymes. Such techniques are widely used in ELISA.

The invention also pertains to a method for following-up a patient (in particular diabetic) for determining presence of peripheral neuropathy, comprising the steps of

(1) determine the level of dp-ucMGP in a patient's sample (blood or plasma)

(2a) if the level of dp-ucMGP is below a predetermined threshold, repeat the surveillance of (1) of a on a regular basis

(2b) if the level of dp-ucMGP is higher than a predetermined threshold, determine the end value from a function as disclosed above

(3a) if the end value of the function is below a given threshold, repeat the surveillance of (1) of a on a regular basis

(3b) if the end value of the function is higher or equal to a given threshold, then make extensive investigation to confirm peripheral neuropathy or provide appropriate treatment.

Surveillance of (2a) and (3a) can be performed every 3 months or every 6 months, but this delay will be adapted by the physician according to the result of the test, but also to the general clinical state of the patient.

The threshold (or reference level) can be selected from

a) an average level of dp-ucMGP obtained from a population of non-diabetic patients; or

b) an average level of dp-ucMGP obtained from a population of diabetic patients that have not developed peripheral neuropathy

c) a mean or median level from a group of individuals having developed peripheral neuropathy (in particular diabetic individuals)

The invention also relates to a method for treating a patient in need thereof, comprising the steps of:

1. Performing the methods are herein disclosed to identify whether the patient has peripheral neuropathy, in particular diabetic peripheral neuropathy, and

2. Applying a treatment regimen to the patient in need thereof according to the presence of peripheral neuropathy, in particular of diabetic peripheral neuropathy.

Treatment of peripheral neuropathy, in particular of diabetic peripheral neuropathy, include tight glucose control, treatments are for reducing pain and other symptoms. There are no real treatments of the disease, and treatment is first intended to decrease pain. Some of the treatment listed below may or may not be effective in all patients.

Medication options for pain control include antiepileptic drugs (AEDs), serotonin-norepinephrine reuptake inhibitors (SNRIs), tricyclic antidepressants (TCAs), and capsaicin cream.

One can also use the antidepressant duloxetine, the anticonvulsant pregabalin, or the long-acting opioid tapentadol ER.

One can also provide AEDs, such as pregabalin.

One can use zonisamide, oxcarbazepine.

One can use serotonin-norepinephrine reuptake inhibitors (SNRIs) such as duloxetine and venlafaxine

One can use selective serotonin reuptake inhibitor such as fluoxetine, paroxetine, sertraline, and citalopram Tricyclic antidepressants

One can use TCAs such as imipramine, amitriptyline, desipramine, and nortriptyline.

It is also possible to use opioid medications, such as oxycodone, tramadol or tapentadol.

One can use topical compositions such as capsaicin applied to the skin in a 0.075% concentration, or more concentrated forms of capsaicin, or clonidine or lidocaine.

It is also foreseen to use botulinum toxin injections, erythropoietin alone or in combination with gabapentin.

Monochromatic infrared photo energy treatment (MIRE) has been shown to be an effective therapy in reducing and often eliminating pain associated with diabetic neuropathy.

Physical therapy is also envisaged, as well as transcutaneous electrical nerve stimulation (TENS) and interferential current (IFC), or exercise programs, that will help to prevent muscle contractures, spasms and atrophy. Heat, therapeutic ultrasound, hot wax are also useful for treating diabetic neuropathy.[

Finally, treatment of early manifestations of sensorimotor polyneuropathy involves improving glycemic control. Tight control of blood glucose can reverse the changes of diabetic neuropathy, but only if the neuropathy and diabetes are recent in onset. The methods herein disclose make it possible to detect early peripheral neuropathy in diabetic patients and thus to either improve the status of the patient via appropriate care or delay onset of invalidating symptoms or progression of the disease.

The invention also relates to an inhibitor of MGP activity for its use for the treatment of a diabetic peripheral neuropathy. In particular, in order to inhibit MGP activity, one can inhibit phosphorylation and/or carboxylation of MGP.

Since carboxylation of MGP is vitamin K dependent, in order to inhibit MGP carboxylation, one can use vitamin K antagonists, or inhibitors of vitamin K epoxide reductase. Thus, one can use 4-hydroxycoumarins (in particular selected from the group consisting of warfarin (Coumadin), coumatetralyl, phenprocoumon, acenocoumarol, dicoumarol, tioclomarol, and brodifacoum). One can also use 1,3-indandione derivatives, in particular selected from the group consisting of pindone, chlorophacinone, diphacinone, anisindione, fluindione and phenindione.

As an example of therapeutic and effective amount, one can use warfarin at a dose comprised between 2 to 10 mg orally once a day.

One could alternatively use an antibiotic that contain a 1-N-methyl-5-thiotetrazole side group (including cefamandole (that can be used at a daily dosage from 500 mg to up to 2 g), moxalactam (that can be used at a daily dosage from 1 g or 15 mg/kg), and cefoperazone (that can be used at a dosage from 2 to 4 grams per day)) or disulfiram (Lipsky, Proc Natl Acad Sci USA. 1984 May; 81(9): 2893-2897).

Obviously, treatment with these compounds should be monitored to reduce the risk of bleeding by the patient.

Alternatively, one can use inhibitors of protein (and especially of serine) phosphorylation. One can thus use serine/threonine kinase inhibitors.

It is to be noted that one can use both an inhibitor of serine/threonine kinase and a vitamin K antagonist or an inhibitor of vitamin K epoxide reductase. These compounds could be used as doses lower than usually used in humans, as it is expected that they would both work in synergy to inhibit activity of MGP while lowering the risks of side effects due to the broad spectrum of actions of the compounds envisaged.

The invention also relates to methods for treating a patient suffering from diabetic peripheral neuropathy, comprising the step of administering a therapeutic amount of an inhibitor of MGP as disclosed above to said patient.

A therapeutic amount is an amount that has a therapeutic effect while minimizing the potential adverse effects. In the present case, the therapeutic effect may be reversal of some of the symptoms of the peripheral neuropathy when it is already present, or to retard apparition of symptoms in a patient presenting an elevated level of dp-ucMGP, or to stop evolution of the disease (stabilize the condition of the patient). The physician shall adjust the dosage of the an inhibitor of MGP in function of the benefit and the side effects observed.

Also subjects of the invention are compositions containing an inhibitor of MGP for the use thereof in the treatment of a diabetic peripheral neuropathy, and also the use of an inhibitor of MGP for the production (manufacture) of a drug intended for the treatment of a diabetic peripheral neuropathy.

### DESCRIPTION OF THE FIGURES

FIG. 1: Schematic description of a method for performing a diagnosis of presence of peripheral neuropathy in a diabetic patient, using the function disclosed in example 3.

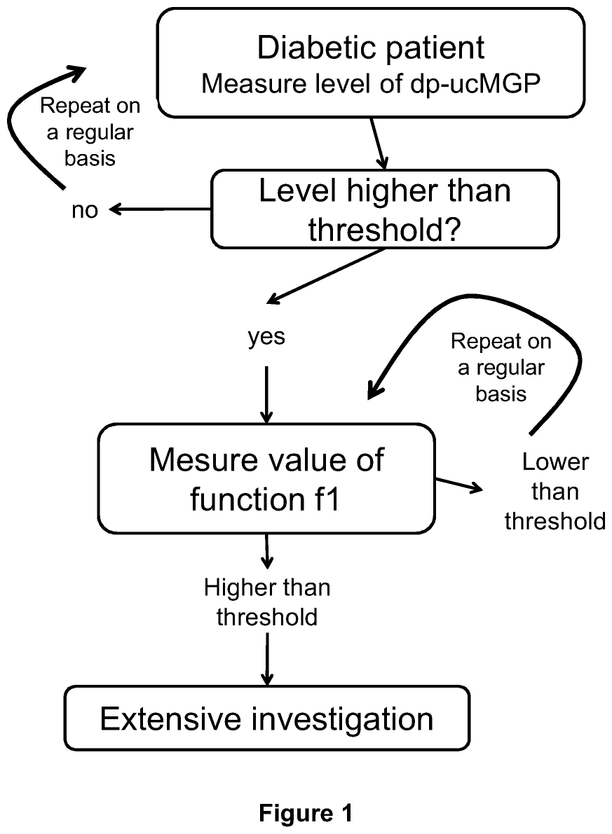

### EXAMPLES

### Example 1. Materials and Methods

Participants

The study focused on patients with type 2 diabetes, at high cardiovascular risk. Inclusion criteria were type 2 diabetes with at least one of the following criteria: coronary artery disease or peripheral arterial occlusive disease or age >50 years for men or >60 years for women. Exclusion criteria were an estimated glomerular filtration rate calculated with the modification of diet in renal disease <30 ml/min and a history of lower limb angioplasty and/or bypass.

Procedure

Data collection, including a clinical evaluation and blood tests, were realized during a one-day hospitalization.

Diabetic Peripheral Neuropathy

Diabetic peripheral neuropathy was assessed by the modified neuropathy disability score (NDS), scoring from 0 to 10 (Young et al., Diabetologia. 1993; 36(2):150-4). NDS assesses vibration sensory on the great toe using 128-Hz tuning fork, temperature sensory on dorsum of the foot using tuning fork with beaker of ice or warm water, pinprick sensory applying pin near to big toe nail and Achilles reflex. Each sensory test scores 0 for normal and 1 for abnormal sensation, for each foot. Achilles reflex score 0 if they are present, 1 if they are present with reinforcement and 2 if they are absent, for each foot. NDS≥6 allows the diagnosis of diabetic peripheral neuropathy (Abbott et al., Diabet Med J Br Diabet Assoc. may 2002; 19(5):377-84). The NDS was also used as a continuous variable to assess peripheral neuropathy severity.

Clinical Data

During the patient interview, the physician collected medical information about personal disease history, comorbidities and treatment. Clinical tests were conducted by a physician blinded to blood tests results.

Biochemical Measures

Blood and urine samples were collected in the morning fasting for the measurement of biochemistry analyses including hemoglobin A1c (HbA1c), high-sensitivity C-reactive protein (hsCRP), estimated glomerular filtration rate (eGFR) by modification of diet in renal disease (MDRD), urinary albumin/creatinine ratio, serum calcium corrected for albumin, serum phosphorus, total cholesterol, triglycerides and IL-6.

Assays were developed to measure two forms of MGP: dephospho-uncardboxylated MGP (dp-uc MGP) and total uncarboxylated MGP (t-uc MGP) (Cranenburg, Thromb Haemost. 2010 October; 104(4):811-22). These assays were conducted after the samples freezing, storage at −80° C. and thawing.

dp-uc MGP levels were measured by a dual-antibody ELISA. The capture antibody was directed against the dephosphorylated MGP sequence 3-15 (mAb-dpMGP; VitaK BV, Maastricht, The Netherlands) and the detecting antibody was directed against the uncarboxylated MGP sequence 35-49 (mAb-ucMGP; VitaK BV). Intra-assay variability was 5.6% for dp-uc MGP and 8.9% for t-uc MGP. Inter-assay variability was 9.9% for dp-uc MGP, and 11.4% for t-uc MGP.

t-uc MGP levels were measured by a competitive, single-antibody ELISA, by an already described process.

dp-uc MGP and t-uc MGP were measured separately in archived samples of 81 age-matched controls. The mean levels were respectively 557+/−277 pmol/l (median: 522 pmol/l), and 4282+/−1100 nmol/l (median: 4109 nmol/l).

Statistical Analyses

Data were described as mean+/−standard deviation of the mean or frequency, as appropriate. Comparison of quantitative variables was performed using Student's t test or Mann-Whitney test, as appropriate. Comparison of qualitative variables was performed using X2 test. Pearson's coefficient (r) was used to assess the variables correlated with NDS, as appropriate. A 95% confidence interval for the correlation coefficient was calculated using Fisher's method (Prism 6; GraphPad Software, Inc). Variables correlated with NDS in univariate analyses were tested in multivariate analysis. Multivariate analysis was performed by analysis of covariance (ANCOVA) or logistic regression, as appropriate (Xlstat software; Addinsoft XLSTAT). A p-value <0.05 was considered statistically significant.

In this cohort (n: 198), the study had a power ≥80% to detect a significant correlation (with r≥0.2, α-risk: 0.05, Student approximation) between each clinical or biological variable and NDS score.

### Example 2. Results

Baseline Characteristics

Clinical and biological characteristics at baseline for the total cohort, and for patients with and without neuropathy are described in Table 1.

TABLE 1
Baseline characteristics

| | | | | | Without | | |
|---|---|---|---|---|---|---|---|
| | | | Neuropathy | | neuropathy | | |
| Characteristics | Total cohort | | (NDS ≥ 6) | | (NDS < 6) | | p-value |
| N | 198 | | 31 | | 167 | | |
| Age, years | 64 ± 8.4 | | 64 ± 8.6 | | 64 ± 8.4 | | ns |
| Male, n(%) | 158 | (80) | 26 | (84) | 132 | (79) | ns |
| Height (cm) | 170 ± 8 | | 173 ± 7 | | 169 ± 8 | | 0.009 |
| Diabetes duration, | 14.6 ± 9.3 | | 14.6 ± 10.2 | | 14.6 ± 9.2 | | ns |
| years | | | | | | | |
| Hypertension, n (%) | 163 | (82) | 28 | (90) | 135 | (81) | ns |
| NDS score, points | 2.4 ± 2.4 | | 6.8 ± 1.5 | | 1.6 ± 1.5 | | <0.0001 |
| Retinopathy treated | 28 | (15) | 10 | (32) | 19 | (11) | 0.003 |
| with laser, n(%) | | | | | | | |
| Coronary arterial | 150 | (76) | 28 | (90) | 122 | (73) | 0.04 |
| disease, n(%) | | | | | | | |
| Ischemic stroke, | 14 | (7) | 2 | (6) | 12 | (7) | ns |
| n(%) | | | | | | | |
| Insulin treatment, | 94 | (47) | 24 | (77) | 70 | (42) | 0.0003 |
| n(%) | | | | | | | |
| eGFR calculated by | 76 ± 20 | | 72 ± 20 | | 77 ± 20 | | ns |
| MDRD, mL/min | | | | | | | |
| Urinary albumin/ | 71 | (36) | 19 | (61) | 52 | (31) | 0.001 |
| creatinine ratio > | | | | | | | |
| 3 (mg/mmol), n(%) | | | | | | | |

| | | | | |
|---|---|---|---|---|
| HbA1c, mmol/mol | 61.8 ± 16.2 | 66.6 ± 20.5 | 60.9 ± 15.2 | ns |
| HbA1c, % | 7.8 ± 1.5 | 8.2 ± 1.9 | 7.7 ± 1.4 | ns |
| hsCRP, mg/L | 2.2 ± 2.5 | 2.4 ± 2.8 | 2.2 ± 2.5 | ns |
| IL-6, pg/mL | 5 ± 22 | 4.6 ± 3.4 | 5.3 ± 24 | ns |
| Corrected calcium, | 2.3 ± 0.1 | 2.3 ± 0.3 | 2.3 ± 0.1 | ns |
| mmol/L | | | | |
| Phosphorus, | 1.02 ± 0.15 | 1.02 ± 0.14 | 1.02 ± 0.16 | ns |
| mmol/L | | | | |
| Triglycerides, | 1.6 ± 1.1 | 1.5 ± 0.8 | 1.6 ± 1.1 | ns |
| mmol/L | | | | |
| Total cholesterol, | 3.7 ± 0.9 | 3.4 ± 0.8 | 3.8 ± 0.9 | 0.02 |
| mmol/L | | | | |
| t-uc MGP, nmol/L | 4868 ± 1613 | 4711 ± 1534 | 4897 ± 1630 | ns |
| dp-uc MGP, pmol/L | 627 ± 451 | 821 ± 703 | 591 ± 379 | 0.009 |

Quantitative variables are represented by mean ± standard deviation. Data are no significant (ns) if p > 0.05. Significant differences between patients with and without neuropathy are in bold.

198 patients were included in the study, of whom 80% of men. The mean age was 64+/−8 years and the mean height was 1.7+/−0.08 meters. Diabetes duration was 15+/−9 years, and mean HbA1c was 7.8%+/−1.5%. Concerning diabetes comorbidities, 15% had a retinopathy treated with laser, 36% of patients had a urinary albumin/creatinine ratio>3 mg/mmol, and mean eGFR calculated by MDRD was 80+/−19 ml/min. Mean NDS was 2.4+/−2.4 points, and 16% of subjects had a diabetic peripheral neuropathy, defined by NDS≥6.

Mean level of dp-uc MGP was 627+/−451 pmol/l, and mean level of t-uc MGP was 4868+/−1613 nmol/l.

Patients with neuropathy were significantly higher (173 cm vs 169 cm, p=0.009).

Dp-uc MGP levels significantly differed in patients with neuropathy from patients without neuropathy (respectively, 821 pmol/l vs 591, p=0.009). Retinopathy treated with laser, urinary albumin/creatinine ratio>3 mg/mmol, coronary arterial disease and insulin treatment were significantly more common in patients with neuropathy. Age, sex ratio, diabetes duration and HbA1c were not different between patients with and without neuropathy.

In univariate analysis (Table 2), NDS is positively associated with height (r=0.25, p=0.0004), HbA1c (r=0.21, p=0.04) and dp-uc MGP (r=0.22, p=0.002). NDS is negatively associated with eGFR (r=−0.16, p=0.03).

TABLE 2
Univariate analysis: correlations between
clinical and biological variables and NDS

| | |
|---|---|
| r, [CI 95%] | p-value |

| | | | |
|---|---|---|---|
| Age | 0.07 | [−0.08; 0.20] | ns |
| Height | 0.25 | [0.11; 0.38] | 0.0004 |
| Diabetes duration | 0.03 | [−0.11; 0.17] | ns |
| eGFR | −0.16 | [−0.29; −0.02] | 0.03 |
| HbA1c | 0.21 | [0.08; 0.34] | 0.04 |
| HsCRP, mg/L | 0.03 | [−0.11; 0.17] | ns |
| IL-6, pg/mL | 0.08 | [−0.07; 0.21] | ns |
| Corrected calcium, mmol/L | 0.08 | [−0.06; 0.22] | ns |
| Phosphorus, mmol/L | 0 | [−0.14; 0.14] | ns |
| Triglycerides | −0.07 | [−0.21; 0.07] | ns |
| Total cholesterol | −0.11 | [−0.25; 0.02] | ns |
| t-uc MGP, nM | −0.07 | [−0.20; 0.07] | ns |
| dp-uc MGP | 0.22 | [0.08; 0.34] | 0.002 |

Correlations were performed by Pearson's coefficient (r). 95% confidence interval of the correlation coefficient was assessed using Fisher's method, and is presented in brackets. Correlations are significant if p < 0.05. Significant results are presented in bold. Quantitative variables are represented by mean ± standard deviation. Data are no significant (ns) if p > 0.05. Significant differences between patients with and without neuropathy are in bold.

In multivariate analysis integrating all significant covariates (in bold, in tables 1 and 2), NDS score was still associated (r=0.51, p<0.0001) with dp-uc MGP levels (β=0.16, p=0.02), height (β=0.29, p<0.0001), HbA1c (β=0.19, p=0.006), insulin treatment (β=0.19, p=0.007), retinopathy treated by laser (β=0.16, p=0.015) and urinary albumin/creatinine ratio>3 mg/mmol (β=0.14, p=0.031) (Table 3).

TABLE 3
Multivariate analysis: correlations between
clinical and biological variables and NDS

| | β, [95% | | |
|---|---|---|---|
| | confidence | Standard | |
| | interval] | deviation | p-value |
| Height | 0.29, [0.16-0.41] | 0.06 | <0.0001 |
| Retinopathy treated | 0.16, [0.03-0.29] | 0.067 | 0.015 |
| with laser | | | |
| Insulin treatment | 0.19, [0.05-0.33] | 0.07 | 0.007 |
| Urinary albumin/ | 0.14, [0.01-0.28] | 0.07 | 0.031 |
| creatinine ratio > 3 | | | |
| HbA1c | 0.19, [0.06-0.33] | 0.07 | 0.006 |
| dp-uc MGP | 0.16, [0.02-0.29] | 0.07 | 0.025 |

Abbreviations: β: standardized coefficient. Multivariate analysis was performed using ANCOVA. 95% confidence interval of the standardized coefficient is presented in brackets. Correlations are significant if p < 0.05. Significant results are presented in bold.

In multivariate analysis integrating all significant covariates (in bold, in tables 1 and 2), presence of neuropathy defined by NDS score ≥6 was still associated (r=0.51, p<0.0001) with dp-uc MGP levels (β=−0.26, p=0.04), height (β=−0.38, p=0.01), insulin treatment (β=0.42, p=0.002), retinopathy treated by laser (β=0.26, p=0.02), and total cholesterol level (β=0.3, p=0.03) (Table 4).

TABLE 4
Multivariate analysis: correlations between clinical and
biological variables and diabetic neuropathy (NDS ≥ 6)

| | β, [95% | | |
|---|---|---|---|
| | confidence | Standard | |
| | interval] | deviation | p-value |
| Height | −0.38, [−0.67-0.09] | 0.15 | 0.01 |
| Retinopathy treated | 0.26, [0.05-0.47] | 0.11 | 0.02 |
| with laser | | | |
| Insulin treatment | 0.42, [0.15-0.7] | 0.14 | 0.002 |
| Total cholesterol | 0.3, [0.03-0.57] | 0.14 | 0.03 |
| dp-uc MGP | −0.26, [−0.51-0.01] | 0.13 | 0.045 |

Abbreviations: β: standardized coefficient. Multivariate analysis was performed using ANCOVA. 95% confidence interval of the standardized coefficient is presented in brackets. Correlations are significant if p < 0.05. Significant results are presented in bold.

### Example 3. Development of a Multivariate Function

A multivariate function was developed:

f1=1/(1+exp(−(−8.600*Height (m)+0.616*Total Cholesterol−1.045*10⁻³*dp-ucMGP(pM)+1.541*[Insulin treatment]+1.312*[retinopathy treated with laser]+0.863*[Albumin/creatinine ratio>3]+12.805))

In this function,

Insulin treatment: in the presence of insulin treatment value=0=yes; if not value=1

retinopathy treated with laser: in the presence of retinopathy treated with laser, value=0; if not, value=1

Albumin/creatinine ratio>3: if Albumin/creatinine ratio higher than 3, the value=0; if Albumin/creatinine ratio≤3, the value is 1.

This function makes it possible to diagnose the presence of peripheral neuropathy with a NDS≥6 (see below).

With a threshold at 0.9595, the function has a sensibility of 35.33% and a specificity of 96.77% the Negative Predictive value is 77.5%.

The AUROC is 0.838. The threshold value was selected to have high VPN and specificity, to exclude patients without peripheral neuropathy. For the patients positive to the test, other investigation may be necessary.

### Example 4. Discussion

Examples 1 and 2 reveal that peripheral neuropathy, defined by a NDS score ≥6, in type 2 diabetic patients is significantly associated with height, insulin treatment, retinopathy treated with laser, total cholesterol and, particularly to dp-uc MGP levels. These factors, HbA1c and urinary albumin/creatinine ratio>3 mg/mmol are also associated with the severity of diabetic neuropathy, defined by NDS.

Height, poor glycemic control and total cholesterol are known risk factors of diabetic neuropathy (Papanas et al., Rev Diabet Stud RDS. Spring-Summer 2015; 12(1-2):48-62). In type 2 diabetes, insulin treatment is required when glycemic control cannot be achieved with oral medications after a few years of disease evolution due to relative insulin deficiency. So it reflects diabetes duration and poor glycemic control, and that is probably why it is associated with diabetic neuropathy in this study. Retinopathy and nephropathy are usual comorbidities of diabetic neuropathy, explaining their association in this study.

So, the results are consistent with other studies and well-known associations with diabetic neuropathy, allowing to consider this cohort as a representative sample of patients with type 2 diabetes and with diabetic neuropathy.

The most important result is the correlation between dp-uc MGP levels and diabetic neuropathy. Moreover, dp-uc MGP levels increase with the severity of diabetic neuropathy.

T-uc MGP levels aren't correlated with diabetic neuropathy.

One hypothesis could be that the association between dp-uc MGP and neuropathy directly results from MGP activity. MGP is a protein from extracellular matrix expressed in osteoarticular and vascular systems. Interestingly Goritz et al have shown that MGP is also expressed by neurons (Goritz, op. cit.), but its role in nervous system remains unclear.

Dosing the level or amount of dp-uc MGP is really useful for clinical practice. Indeed, the diagnostic of diabetic neuropathy is mainly clinical, based on sensory tests, but these tests need to be associated to increase their sensitivity, are operator-dependent and time-consuming. Different surveys revealed that about only 65% of patients with diabetes yearly had a foot examination by a physician.

It is thus interesting, for clinical practice, to have a biomarker of diabetic neuropathy.

Several biomarkers had been suggested, as neuron-specific enolase, toll-like receptor 4 or (TNF-α), but they are not specific of diabetic neuropathy (Zhu et al., Neurosci Lett. 12 janv 2015; 585:28-32; Li et al., Diabetes Care. nov 2013; 36(11):3405-10).

### Example 5. Treatment of Diabetic Peripheral Neuropathies

The pathogenesis of diabetic neuropathy remains unclear.

Oxidative stress related to chronic hyperglycemia is one of the main hypothesis developed during the last decades. Excess of intracellular glucose overloads the glycolysis. This results in the activation of polyols, hexosamine and protein kinase C pathways and in the generation of advanced glycation end products. These pathways generate reactive oxygen species, reduce NADPH levels and trigger inflammatory signaling cascade, leading to nerve damage.

Targeted therapies have been developed: aldose reductase inhibitors, blocking the polyol pathway, protein kinase C inhibitors, and aminoguanidine, can be used, preventing the synthesis of age glycation end products. Despite promising results in pre-clinical animal models, the benefit in man was however limited in clinical trials.

Only α-lipoic acid, an antioxidant treatment, seems to be slightly efficient and safe for clinical use in diabetic neuropathy.

Thus, the treatment of diabetic neuropathy remains mainly symptomatic, based on pain treatment.

The results reported above show that MGP may be implicated in the pathophysiology of diabetic neuropathy. Targeted therapies on MGP are thus to be developed.

Since MGP is a vitamin K dependent protein, vitamin K could be the target of therapies. Warfarin, which is an inhibitor of vitamin K epoxide reductase, inhibits the activity of MGP.

In view of the fact that it has been reported that improvement of neuropathic symptoms and pain has been reported after warfarin administration (Henry Gomez et al., BMJ Case Rep. 17 juin 2016; 2016), such product and other antivitamin K products are good and plausible candidates to be used for treatment of diabetic peripheral neuropathy.

1. A method for determining the presence of peripheral neuropathy in a patient comprising:
a) determining the level of dephosphorylated uncarboxylated Matrix gla protein (MGP) in a sample from said patient and optionally of other clinical markers;
b) when other markers are used, combining the values obtained in a) through a function in order to obtain an end result;
c) comparing the level of dephosphorylated uncarboxylated MGP measured in step a) or of the end value obtained in step b) to a predetermined threshold;
wherein the patient has peripheral neuropathy if the level of dephosphorylated uncarboxylated MGP measured in step a) or of the end value obtained in step b) is higher than the threshold.

2. The method of claim 1, wherein the peripheral neuropathy is a diabetic peripheral neuropathy.

3. The method of claim 1, wherein other clinical markers are obtained from the patient, preferably selected from the group consisting of height of the patient (m), total cholesterol (mmol/l), presence of insulin treatment (yes=0; no=1), presence of retinopathy treated with laser (yes=0; no=1), presence of a albumin/creatinine (mg/mmol) ratio>3 ((yes=0; no=1)), glycated hemoglobin (HbA1c) and presence of coronary arterial disease (yes=0; no=1).

4. The method of claim 3, wherein the function of step b) is f1=1/(1+exp(−(−a*Height (m)+b*Total Cholesterol−c*dp-ucMGP(pM)+d*[Insulin treatment]+e*[retinopathy treated with laser]+f*[Albumin/creatinine ratio>3]+g)), with
8.3≤a≤8.9, more preferably 8.4≤a≤8.8, most preferably 8.5≤a≤8.7
0.45≤b≤0.75, more preferably 0.5≤b≤0.7, most preferably 0.55≤b≤0.65
1.02×10⁻³≤c≤1.06×10⁻³, more preferably 1.035×10⁻³≤c≤1.055×10⁻³, most preferably 1.04×10⁻³≤c≤1.05×10⁻³,
1.3≤d≤1.8, more preferably 1.4≤d≤1.7, most preferably 1.5≤4≤1.6
1.05≤e≤1.55, more preferably 1.15≤e≤1.45, most preferably 1.25≤e≤1.35
0.6≤f≤1.1, more preferably 0.7≤f≤1.0, most preferably 0.8≤f≤0.9 and
12.4≤g≤13.2, more preferably 12.6≤f≤13.0, most preferably 12.7≤f≤12.9.

5. The method of claim 4, wherein
a=8.600,
b=0.616,
c=1.045×10−3,
d=1.541,
e=1.312,
f=0.863, and
g=12.805

6. The method of claim 1, wherein the level of dephosphorylated uncarboxylated MGP in step a) is measured by ELISA assay.

7-9. (canceled)

10. The method of claim 1, wherein the patient does not have peripheral neuropathy when the level of dephosphorylated uncarboxylated MGP is below 700 pmol/l.

11. The method of claim 5, wherein the patient does not have peripheral neuropathy when the value of the function is below 0.9595.

12. The method of claim 1, wherein the patient is a type 2 diabetic patient.

13. The method of claim 1, wherein the neuropathy is defined by a NDS (Neuropathy Disability Score) higher than 6.

14-16. (canceled)

17. A method for treating a patient in need thereof, comprising:
a) determining the level of dephosphorylated uncarboxylated Matrix gla protein (MGP) in a sample from said patient and optionally of other clinical markers;
b) when other markers are used, combining the values obtained in a) through a function in order to obtain an end result; and
c) comparing the level of dephosphorylated uncarboxylated MGP measured in step a) or of the end value obtained in step b) to a predetermined threshold;
wherein the patient has peripheral neuropathy if the level of dephosphorylated uncarboxylated MGP measured in step a) or of the end value obtained in step b) is higher than the threshold, and
d) treating the patient when the patient is detected as having peripheral neuropathy.

18. The method of claim 17, wherein the peripheral neuropathy is a diabetic peripheral neuropathy.

19. The method of claim 17, wherein other clinical markers are obtained from the patient and are preferably selected from the group consisting of height of the patient (m), total cholesterol (mmol/l), presence of insulin treatment (yes=0; no=1), presence of retinopathy treated with laser (yes=0; no=1), presence of a albumin/creatinine (mg/mmol) ratio>3 ((yes=0; no=1)), glycated hemoglobin (HbA1c), and presence of coronary arterial disease (yes=0; no=1).

20. The method of claim 19, wherein the function of step b) is f1=1/(1+exp(−(−a*Height (m)+b*Total Cholesterol−c*dp-ucMGP(pM)+d*[Insulin treatment]+e*[retinopathy treated with laser]+f*[Albumin/creatinine ratio>3]+g)), with
8.3≤a≤8.9, more preferably 8.4≤a≤8.8, most preferably 8.5≤a≤8.7
0.45≤b≤0.75, more preferably 0.5≤b≤0.7, most preferably 0.55≤b≤0.65
1.02×10⁻³≤c≤1.06×10⁻³, more preferably 1.035×10⁻³≤c≤1.055×10⁻³, most preferably 1.04×10⁻³≤c≤1.05×10⁻³,
1.3≤d≤1.8, more preferably 1.4≤d≤1.7, most preferably 1.5≤4≤1.6
1.05≤e≤1.55, more preferably 1.15≤e≤1.45, most preferably 1.25≤e≤1.35
0.6≤f≤1.1, more preferably 0.7≤f≤1.0, most preferably 0.8≤f≤0.9 and
12.4≤g≤13.2, more preferably 12.6≤f≤13.0, most preferably 12.7≤f≤12.9.

21. The method of claim 20, wherein
a=8.600,
b=0.616,
c=1.045×10⁻³,
d=1.541,
e=1.312,
f=0.863, and
g=12.805.

22. The method of claim 17, wherein the level of dephosphorylated uncarboxylated MGP in step a) is measured by ELISA assay.

23. The method of claim 17, wherein the patient does not have peripheral neuropathy when the level of dephosphorylated uncarboxylated MGP is below 700 pmol/l.

24. The method of claim 21, wherein the patient does not have peripheral neuropathy when the value of the function is below 0.9595.

25. The method of claim 17, wherein the patient is a type 2 diabetic patient.

26. The method of claim 17, wherein the neuropathy is defined by a NDS (Neuropathy Disability Score) higher than 6.

27. The method of claim 17, wherein an inhibitor of MGP activity is administered to the patient.

28. The method of claim 27, wherein the inhibitor of MGP activity is selected in the group consisting of an anti-vitamin K, an inhibitor of vitamin K epoxide reductase, and warfarin.
